(19) United States
(12) Patent Application Publication
(10) Pub. No.: US 2021/0113179 A1
(43) Pub. Date: Apr. 22, 2021

(54) METHODS OF ENHANCING THE ACCURACY AND/OR SENSITIVITY OF ULTRASOUND IMAGING IN DIAGNOSING TUMORS

(71) Applicants: Academia Sinica, Taipei (TW); National Chiao Tung University, Hsinchu City (TW)

(72) Inventors: Chung-Hsuan CHEN, Taipei (TW); Shok-Li NG, Taipei (TW); Yung-Chieh CHAN, Taipei (TW); Peter LAI, Taipei (TW); Michael HSIAO, Taipei (TW); Olga K KOSHELEVA, Taipei (TW); Nelson G. CHEN, Hsinchu City (TW)

(73) Assignees: Academia Sinica, Taipei (TW); National Chiao Tung University, Hsinchu City (TW)

(21) Appl. No.: 17/073,408

(22) Filed: Oct. 19, 2020

Related U.S. Application Data
(60) Provisional application No. 62/916,768, filed on Oct. 17, 2019.

(51) Int. Cl. A61B 8/08; A61K 49/22

(52) U.S. Cl. CPC A61B 8/0833; A61B 8/5215; A61K 49/226

(57) ABSTRACT

Disclosed herein is a method of enhancing the accuracy and/or sensitivity of ultrasound imaging in detecting a tumor in a subject. The method comprises administering to the subject an effective amount of a nanoparticle prior to the application of ultrasound to the subject. According to certain embodiments of the present disclosure, the nanoparticle is a magnetic nanoparticle, for example, a gold, silver, or iron oxide nanoparticle. Also disclosed herein are methods of treating a tumor in a subject by detecting the tumor via ultrasound with the aid of a nanoparticle, and then administering to the subject an anti-cancer treatment based on the location of the tumor revealed by the ultrasound image.

### CROSS-REFERENCE TO RELATED APPLICATIONS

This application relates to and claims the benefit of U.S. Provisional Application No. 62/916,768, filed Oct. 17, 2019; the content of the application is incorporated herein by reference in its entirety.

### BACKGROUND OF THE INVENTION

### 1. Field of the Invention

The present disclosure in general relates to the field of tumor diagnosis and treatment. More particularly, the present disclosure relates to methods of detecting a tumor in a subject by use of ultrasound with the aid of a nanoparticle, followed by administering to the subject an anti-cancer treatment in accordance with the location of the tumor revealed by the ultrasound image.

### 2. Description of Related Art

Cancer is a group of diseases caused by abnormal cell growth. According to the report of World Health Organization (WHO), cancer is the second leading cause of death globally that is responsible for an estimated 9.6 million deaths in 2018. Common cancers include lung cancer, colorectal cancer, stomach cancer (also known as gastric cancer), liver cancer, and breast cancer, which collectively lead to 4.8 million deaths in 2018. Early diagnosis is crucial to improve the survival rates of cancer patients. Compared with late-stage cancers, early-stage cancers are more responsive to anti-tumor treatments.

Common approaches for diagnosing cancers include computed tomography (CT) scan, magnetic resonance imaging (MRI), and ultrasound imaging. However, none of these approaches provides a satisfactory effect. For example, CT scan involves exposure to intense ionizing radiation, which may cause cell death and/or chromosomal aberrations via ionizing cellular components, such as cell membrane, organelle, and DNA molecule. MRI is a very expensive and time consuming procedure. Further, MRI is not applicable to the patient suffering from claustrophobia or anxious due to the small and enclosed patient bore, and the patient implanted with metallic foreign bodies. Regarding ultrasound imaging, due to its limited resolution, it is only useful in detecting tumors having a diameter usually greater than 1 cm, but not early-stage tumors that usually are less than 1 cm in size.

In view of the foregoing, there exists in the related art a need for a novel method for making a diagnosis of tumors, especially for diagnosing the early-stage.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present disclosure is directed to the use of nanoparticles in assisting the ultrasound imaging for detecting tumors, thereby improving the efficacy of ultrasound imaging in diagnosing and/or treating tumors, especially the early-stage tumors.

Accordingly, the first aspect of the disclosure is directed to a method of enhancing the accuracy and/or sensitivity of ultrasound imaging of a tumor in a subject by use of a nanoparticle. The method comprises,
(a) administering to the subject an effective amount of a nanoparticle; and
(b) applying ultrasound to the subject of the step (a) to produce an ultrasound image of the tumor and a neighboring normal tissue, wherein the nanoparticle increases the contrast between the tumor and the normal tissue on the ultrasound image thereby enhancing the accuracy and/or sensitivity of ultrasound imaging in detecting the tumor.

Also disclosed herein are methods of diagnosing and/or treating tumors by ultrasound with the aid of a nanoparticle. The method of making a diagnosis of a tumor in a subject comprises the steps of,
(a) administering to the subject an effective amount of a nanoparticle;
(b) applying ultrasound to the subject of the step (a) so as to produce an ultrasound image of the subject; and
(c) determining the presence or absence of the tumor based on the ultrasound image produced in the step (b) thereby making the diagnosis of the tumor.

The method of treating a tumor in a subject comprises the steps of,
(a) administering to the subject an effective amount of a nanoparticle;
(b) applying ultrasound to the subject of the step (a) so as to produce an ultrasound image of the tumor, in which the location of the tumor is revealed by the ultrasound image; and
(c) administering to the subject a therapeutically effective amount of a radiation or an anti-tumor agent based on the location of the tumor revealed by the ultrasound image in the step
(b).

Another aspect of the present disclosure provides a method of diagnosing and treating a tumor in a subject. The method comprises,
(a) administering to the subject an effective amount of a nanoparticle;
(b) applying ultrasound to the subject of the step (a) so as to produce an ultrasound image of the subject;
(c) determining the presence or absence of the tumor based on the ultrasound image produced in the step (b); and
(d) administering to the subject a therapeutically effective amount of a radiation or an anti-tumor agent based on the result determined in the step (c), wherein the subject has the tumor.

Also disclosed herein is a use of a nanoparticle in the preparation of a contrast enhancer for enhancing the accuracy or sensitivity of ultrasound imaging in the detection and treatment of a tumor in a subject. According to some embodiments of the present disclosure, the a radiation or an anti-tumor agent is administered to the subject based on the location of the tumor revealed by the ultrasound imaging.

According to embodiments of the present disclosure, the nanoparticle employed in the methods of the present disclosure may be a metal nanoparticle, a metal oxide nanoparticle, or an organic nanoparticle (such as liposome, polystyrene and etc.). Non-limiting examples of the metal nanoparticle suitable to be employed in the present methods include, silver (Ag) nanoparticle, gold (Au) nanoparticle, platinum (Pt) nanoparticle, palladium (Pd) nanoparticle, copper (Cu) nanoparticle, cobalt (Co) nanoparticle, chromium (Cr) nanoparticle, nickel (Ni) nanoparticle, iron (Fe) nanoparticle, titanium (Ti) nanoparticle, aluminum (Al) nanoparticle, lead (Pb) nanoparticle, rhodium (Rh) nanoparticle, tantalum (Ta) nanoparticle, ruthenium (Ru) nanoparticle, tungsten (W) nanoparticle, gadolinium (Gd) nanoparticle, and an alloy thereof. Exemplary metal oxide nanoparticle suitable to be employed in the present methods include, but are not limited to, zinc oxide (ZnO) nanoparticle, magnesium oxide (MgO) nanoparticle, manganese oxide (Mn₂O₃) nanoparticle, magnetite (Fe₃O₄) nanoparticle, maghemite (γ-Fe₂O₃) nanoparticle, cobalt ferrite (CoFe₂O₄) nanoparticle, manganese ferrite (MnFe₂O₄) nanoparticle, and strontium iron oxide (SrFe₁₂O₁₉) nanoparticle. Alternatively, the nanoparticle employed in the methods of the present disclosure may be a silicon (Si) nanoparticle or a silicon oxide nanoparticle.

In the present methods, the diameter of the nanoparticle may vary with desired purposes. Preferably, the diameter of the nanoparticle of the present disclosure ranges from 1 nanometer (nm) to 999 nanometers.

Optionally, the nanoparticle of the present disclosure further comprises an antibody or a fragment thereof, an aptamer, a targeting peptide conjugated thereto.

The nanoparticle of the present method may be administered to the subject via any suitable route, for example, parenteral injection, intravenous injection, intra-arterial injection, intramuscular injection, or subcutaneous injection. Depending on the administration route, the nanoparticle may be formulated and administered in a solution, an emulsion, or a gel.

According to some embodiments of the present disclosure, the nanoparticle is administered in the solution, and the nanoparticle is present in the solution at a concentration of 1 mg/ml to 100 mg/ml. In certain embodiments, the solution is administered to the subject in a volume of 0.01 ml/Kg to 50 ml/Kg body weight of the subject.

Depending on desired purposes, the effective amount of the nanoparticle administered to the subject is about 0.01-1,000 mg/Kg; preferably, about 0.1-100 mg/Kg. More preferably, the dose of about 1-10 mg/Kg of the nanoparticle is sufficient to achieve the enhancing effect on ultrasound imaging.

The tumor detected by and/or treatable with the method of the present disclosure may be melanoma, tongue carcinoma, colorectal carcinoma, esophageal carcinoma, gastric carcinoma, lung cancer, bladder cancer, breast cancer, pancreatic cancer, renal cancer, hepatocellular carcinoma, ovarian cancer, prostate cancer, or head and neck squamous cell carcinoma.

The anti-tumor agent for treating the tumor as described in the present method may be any of a chemotherapeutic agent, an anti-proliferative agent, an anti-angiogenic agent, an immunomodulatory agent, or an anti-hormone agent.

The subject is a mammal; preferably, a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A-1D are ultrasound images of capillary tubing embedded in agarose phantom gel. The capillary tubing was injected with water or Fe₃O₄ nanoparticle according to Example 1.1 of the present disclosure. FIG. 1A: B-mode (brightness mode) images of capillaries, which are respectively injected with water (ddH₂O, the left photograph) and Fe₃O₄ nanoparticle (the right photograph). FIG. 1B: harmonic images of capillaries, which are respectively injected with water (ddH₂O, the left panel) and Fe₃O₄ nanoparticle (the right photograph). FIG. 1C: cross-sectional views of B-mode image (the left panel) and B-mode+harmonic image (the right photograph) of capillaries. FIG. 1D: cross-sectional views of B-mode image of capillaries.

FIG. 2 is an ultrasound image of agar phantom gel injected with water (ddH₂O, indicated by the left arrow) or silver nanoparticle (AG, indicated by the right arrow) according to Example 1.2 of the present disclosure.

FIG. 3 is an ultrasound image of tissue-mimicking phantom, where artificial tumors soaked in water (DDH₂O, indicated by the left arrows) or Fe₃O₄ nanoparticle (MNP, indicated by the right arrows) were embedded, according to Example 1.3 of the present disclosure.

FIG. 4 is an ultrasound image of 2% agar phantom, where artificial tumors soaked in water (DDH₂O, indicated by the left arrow) or Fe₃O₄ nanoparticle (MNP, indicated by the right arrow) were embedded, according to Example 1.4 of the present disclosure.

FIGS. 5A-5D are ultrasound images of pork injected with phosphate-buffered saline (PBS) (FIG. 5A), gold nanoparticle (FIG. 5B), or Fe₃O₄ nanoparticle (FIG. 5C: 0.03077 mg/ml;

FIG. 5D: 5 mg/ml) according to Example 2 of the present disclosure. Left photographs: before injection; right photographs: after injection. The injected site was marked by the arrow and/or circle.

FIGS. 6A-6C are ultrasound images of tumors injected with PBS (FIG. 6A), Fe₃O₄ nanoparticle (FIG. 6B) or gold nanoparticle (FIG. 6C) according to Example 3.1 of the present disclosure. Left photographs: before injection; right photographs: after injection.

FIGS. 7A and 7B are ultrasound images of tumors before (FIG. 7A) and after (FIG. 7B) the injection of Fe₃O₄ nanoparticle according to Example 3.2 of the present disclosure.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example.

However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “nanoparticle” as used herein refers to any particle that is on the order of 10⁻⁹ meter, or one billionth of a meter. Specifically, the term “nanoparticle” refers to any particle having a diameter of less than 1,000 nanometer (nm). In various embodiments, the nanoparticle has a characteristic size (e.g., diameter or edge side) less than about 1,000 nm, 800 nm, or 500 nm; preferably less than about 400 nm, 300 nm, or 200 nm; more preferably about 100 nm or less, about 50 nm or less, or about 30 or 20 nm or less. Each nanoparticles can comprise a core, a core and a shell (as in core-shell nanoparticles), or a core and multiple shells (as in core-multi-shell particles).

The term “normal tissue” as used herein refers to a non-cancerous tissue, i.e., a healthy and non-malignant tissue. Specifically, the term “normal tissue” refers to a tissue that has no apparent growth abnormalities or other sign of carcinoma, neoplasia or dysplasia, e.g., loss of ploidy, dedifferentiation, metastasis and/or tissue invasion capability. As used herein, the term “neighboring normal tissue” refers to a normal tissue (i.e., a non-cancerous tissue) that is adjacent or in proximity to the tumor.

The term “diagnosis” as used herein refers to methods by which a skilled artisan can estimate and/or determine the probability (“a likelihood”) of whether a patient is suffering from a given disease, disorder, or condition, for example, tumors. The diagnostic method of the present invention may be used independently, or in combination with other diagnostic and/or staging methods known in the medical art for a specific disease, disorder, or condition. That such a diagnosis is “determined” is not meant to imply that the diagnosis is 100% accurate.

As used herein, the term “treatment” includes preventative (e.g., prophylactic), curative or palliative treatment of a disease (e.g., a cancer) in a mammal, particularly human; and includes: (1) preventative (e.g., prophylactic), curative or palliative treatment of a disease or condition (e.g., a tumor) from occurring in an individual who may be pre-disposed to the disease but has not yet been diagnosed as having it; (2) inhibiting a disease (e.g., by arresting its development and/or process); or (3) relieving a disease (e.g., reducing symptoms associated with the disease).

The term “administered”, “administering” or “administration” are used interchangeably herein to refer a mode of delivery, including, without limitation, parenterally, intravenously, intra-arterially, intramuscularly, and subcutaneously administering an agent (e.g., a nanoparticle) of the present invention.

The term “contrast” has the conventional meaning in the field of in vivo medical imaging, and refers to the relative difference of signal or echo intensities in two adjacent regions of an image. As used herein, the term “contrast” refers to the relative difference of signal or echo intensities between the cancerous tissue and its neighboring normal tissues on a ultrasound image. The term “contrast enhancer” as used herein refers to a material or a substance (e.g., the nanoparticle of the present disclosure) that enhances the relative difference of signal or echo intensities between the cancerous tissue and its neighboring normal tissues on a ultrasound image, and thus, improving the intensity discernment between the cancerous and normal tissues.

The term “effective amount” as referred to herein designate the quantity of a component which is sufficient to yield a desired response or effect, for example, increasing the contrast between normal tissues and tumors on ultrasound image thereby enhancing the accuracy and/or sensitivity of ultrasound imaging in distinguishing tumors from surrounding normal tissues. The specific effective or sufficient amount will vary with such factors as the type or size of the tumor, the physical condition of the patient (e.g., the patient's body mass, age, or gender), the type of mammal or animal being administered, the parameters (e.g., intensity or frequency) of the ultrasound administered to the subject, and the formulation of the nanoparticle. Effective amount may be expressed, for example, in grams, milligrams or micrograms or as milligrams per kilogram of body weight (mg/Kg). Alternatively, the effective amount can be expressed in the concentration of an agent (e.g., the nanoparticle), such as molar concentration, mass concentration, volume concentration, molality, mole fraction, mass fraction and mixing ratio. Persons having ordinary skills could calculate the human equivalent dose (HED) for the agent (such as the nanoparticle) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

Unless otherwise indicated, a “therapeutically effective amount” of a radiation or an anti-tumor agent is an amount sufficient to provide a therapeutic benefit in the treatment or management of a disease or condition (e.g., a tumor), or to delay or minimize one or more symptoms associated with the disease or condition. A therapeutically effective amount of a radiation or an anti-tumor agent is an amount of therapeutics, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

The term “subject” or “patient” refers to a mammal including the human species that may be diagnosed and/or treated by the method of the present invention. The term “subject” is intended to refer to both the male and female gender unless one gender is specifically indicated.

### II. Description of the Invention

The present disclosure is based, at least in part, on the discovery that certain nanoparticles are useful in enhancing the accuracy and/or sensitivity of ultrasound imaging of tumors. Specifically, the inventors of the present disclosure unexpectedly discovered that once being administered to a cancer patient (i.e., an individual suffering from a cancer), these nanoparticles tend to be trapped and accumulated in the tumor due to the highly porous structure of tumors; such accumulation of nanoparticles in cancerous tissue leads to enhanced contrast between the cancerous tissue and its neighboring normal tissues (i.e., non-cancerous tissues surrounding or being adjacent to the cancerous tissue) in the ultrasound images. Accordingly, nanoparticles may serve as a contrast enhancer to enhance the accuracy and/or sensitivity of ultrasound imaging in detecting tumors, and based on such an enhanced ultrasound image, one skilled artisan may accurately determine the presence or absence, and/or the location of the cancerous tissue (i.e., tumor) in the subject, and administer to the subject an appropriate treatment, for example, a radiation or an anti-tumor agent.

The first aspect of the present disclosure thus is directed to a method of enhancing the accuracy and/or sensitivity of ultrasound imaging of a tumor in a subject by use of a nanoparticle. The method comprises the steps of,
(a) administering to the subject an effective amount of a nanoparticle; and
(b) applying ultrasound to the subject of the step (a) to produce an ultrasound image of the tumor and a neighboring normal tissue (i.e., a normal tissue surrounding the tumor), wherein the nanoparticle increases the contrast between the tumor and the normal tissue on the ultrasound image thereby enhancing the accuracy and/or sensitivity of ultrasound imaging in detecting the tumor.

In the step (a), a sufficient amount of nanoparticles is administered to a subject (e.g., a subject having or suspected of having a tumor). In general, the subject is a mammal; for example, a human, a mouse, a rat, a hamster, a guinea pig, a rabbit, a dog, a cat, a cow, a goat, a sheep, a monkey, and a horse. Preferably, the subject is a human. Exemplary tumors suitable to be imaged by the present method include, but are not limited to, melanoma, tongue carcinoma, colorectal carcinoma, esophageal carcinoma, gastric carcinoma, lung cancer, bladder cancer, breast cancer, pancreatic cancer, renal cancer, hepatocellular carcinoma, ovarian cancer, prostate cancer, and head and neck squamous cell carcinoma.

According to certain embodiments of the present disclosure, the subject is a mouse, in which the nanoparticle is administered to the subject in an amount of about 0.1-10,000 mg/Kg; for example, 0.1, 0.2, 0.3, 0.4, 0.5, 0.6, 0.7, 0.8, 0.9, 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1,000, 2,000, 3,000, 4,000, 5,000, 6,000, 7,000, 8,000, 9,000, or 10,000 mg/Kg. According to the preferred embodiments, the nanoparticle is administered to the subject in an amount of about 1-1,000 mg/Kg. In some working examples, about 1-100 mg/Kg is sufficient to enhance the accuracy and/or sensitivity of ultrasound imaging in detecting the tumor. According to one specific example, the nanoparticle is administered to the subject in an amount of 4 mg/Kg.

A skilled artisan could calculate the human equivalent dose (HED) of the nanoparticle to be administered onto the human subject, based on the doses determined from animal models. Accordingly, the effective amount of the nanoparticle suitable for use in a human subject may be in the range of 0.01-1,000 mg/Kg; such as, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 mg/Kg. Preferably, about 0.1-100 mg/Kg is administered to the human subject. According to some specific examples of the present disclosure, the effective amount of the nanoparticle is about 0.1-10 mg/Kg.

The nanoparticle useful for imaging a tumor via ultrasound is preferably a magnetic nanoparticle (MNP), for example, a metal nanoparticle or a metal oxide nanoparticle or an organic nanoparticle. Exemplary metal nanoparticles suitable for use in the present disclosure include, but are not limited to, silver (Ag) nanoparticle, gold (Au) nanoparticle, platinum (Pt) nanoparticle, palladium (Pd) nanoparticle, copper (Cu) nanoparticle, cobalt (Co) nanoparticle, chromium (Cr) nanoparticle, nickel (Ni) nanoparticle, iron (Fe) nanoparticle, titanium (Ti) nanoparticle, aluminum (Al) nanoparticle, lead (Pb) nanoparticle, rhodium (Rh) nanoparticle, tantalum (Ta) nanoparticle, ruthenium (Ru) nanoparticle, tungsten (W) nanoparticle, gadolinium (Gd) nanoparticle, and an alloy thereof. Examples of the metal oxide nanoparticle suitable for use in the present disclosure include, but are not limited to, zinc oxide (ZnO) nanoparticle, magnesium oxide (MgO) nanoparticle, manganese oxide (Mn₂O₃) nanoparticle, magnetite (Fe₃O₄) nanoparticle, maghemite (γ-Fe₂O₃) nanoparticle, cobalt ferrite (CoFe₂O₄) nanoparticle, manganese ferrite (MnFe₂O₄) nanoparticle, and strontium iron oxide (SrFe₁₂O₁₉) nanoparticle. According to one embodiment of the present disclosure, the nanoparticle is a silver nanoparticle. According to another embodiment of the present disclosure, the nanoparticle is a magnetite nanoparticle. According to still another embodiment of the present disclosure, the nanoparticle is a gold nanoparticle. Alternatively, the nanoparticle useful for imaging a tumor via ultrasound may be a silicon nanoparticle or a silicon oxide nanoparticle.

The diameter and the shape of the nanoparticle may vary with intended uses. The diameter of the nanoparticle preferably ranges from 1 nanometer to 999 nanometers; more preferably, ranging from 1 nanometer to 100 nanometer. According to one embodiment, the diameter of the nanoparticle is about 20 nanometers. According to another embodiment, the diameter of the nanoparticle is about 30 nanometers. According to still another embodiment, the diameter of the nanoparticle is about 80 nanometers. The nanoparticle may be shaped as a polygon (for example, octahedral shape), a needle, a dumbbell, a rod, a sphere, a pyramid, or a cylinder. In certain embodiments of the present disclosure, the nanoparticle is shaped as a sphere.

Optionally, the nanoparticle further comprises an antibody or a fragment thereof, an aptamer, a targeting peptide conjugated thereto so as to enhance the efficacy of the nanoparticle in targeting tumor cells.

Depending on the practical situation (e.g., the condition of the subject, the type of tumor, or the portion of the subject's body being examined), the nanoparticle may be administered to the subject via parenteral injection, intravenous injection, intra-arterial injection, intramuscular injection, or subcutaneous injection.

As would be appreciated, the nanoparticle may be formulated and administered in a solution, emulsion, or gel in accordance with the administration route. A pharmaceutical composition of the nanoparticle can be made in a pharmaceutically acceptable vehicle or carrier, such as PBS, sodium chloride solution, Ringer's solution, dextrose solution, sterile water, cream, ointment, lotion, oil, paste and solid carrier.

According to some embodiments of the present disclosure, the nanoparticle is administered in a solution, in which the nanoparticle is present in the solution at a concentration of 1 mg/ml to 100 mg/ml; for example, 1, 2, 3, 4, 5, 6, 7, 8, 9, 10, 15, 20, 25, 30, 35, 40, 45, 50, 55, 60, 65, 70, 75, 80, 85, 90, 95 or 100 mg/ml. In one working example, the nanoparticle is present in the solution at a concentration of 5 mg/ml. In another working example, the nanoparticle is present in the solution at a concentration of 10 mg/ml.

According to certain embodiments of the present disclosure, the solution containing the nanoparticle is administered to the subject in accordance with the body weight of the subject. In some preferred embodiments, the solution is administered to the subject in a volume of 0.01 ml/Kg to 50 ml/Kg body weight of the subject; e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45 or 50 ml/Kg body weight of the subject. More preferably, the solution is administered to the subject in a volume of 0.1 ml/Kg to 20 ml/Kg body weight of the subject. According to some working example, about 0.1 ml/Kg to 10 ml/Kg of the solution containing the nanoparticle is enough to enhance the accuracy and/or sensitivity of ultrasound imaging in the subject.

Alternatively, the nanoparticle may be administered to the subject in accordance with the body surface area of the subject. According to certain embodiments, the nanoparticle is administered to the subject at a concentration of 50 mg nanoparticle/cm³ to 250 mg nanoparticle/cm³; for example, 50, 55, 60, 65, 70, 75, 80, 85, 90, 95, 100, 110, 120, 130, 140, 150, 160, 170, 180, 190, 200, 210, 220, 230, 240 or 250 mg nanoparticle/cm³.

Then, in the step (b), the subject administered with the nanoparticle in the step (a) is subjected to an ultrasound examination (also known as ultrasonography) to produce one or more ultrasound images of the subject. The operational parameters of the ultrasound (e.g., the mode of operation, frequency, intensity, duty cycle, sonication duration, and time of intermittent imaging) may vary with factors such as the nanoparticle administered in the step (a), the type of tumor, the portion of the subject's body being examined, the type of mammal or animal being applied, and the physical condition of the subject. A skilled artisan or a clinical practitioner may adjust the operational parameters of the ultrasound based on the factors described above so as to optimize the visibility (i.e., the accuracy and/or sensitivity) of the thus-produced ultrasound image(s).

Based on the ultrasound image(s), a skilled artisan or a clinical practitioner may determine the presence or absence, or the location (if any) of the tumor, and accordingly, providing the patients with appropriate treatments in time.

The second aspect of the present disclosure thus pertains to a method of making a diagnosis of a tumor in a subject by use of ultrasound with the aid of a nanoparticle. The method comprises,
(a) administering to the subject an effective amount of a nanoparticle;
(b) applying ultrasound to the subject of the step (a) so as to produce an ultrasound image of the subject; and
(c) determining the presence or absence of the tumor based on the ultrasound image produced in the step (b) thereby making the diagnosis of the tumor.

Steps (a) and (b) are the same as those described in the method of the first aspect of the present disclosure; and hence, detailed description of these steps is omitted for the sake of brevity.

As mentioned above, the nanoparticle would be trapped and accumulated in the cancerous tissue rather than the normal tissue, thereby creating an enhanced contrast between the tumor and its neighboring normal tissues on the ultrasound images. Accordingly, the presence or absence of the tumor may be detected in a more accurate and precise manner based on the enhanced contrast on the ultrasound images (step (c)).

Also disclosed in the present disclosure is a method of treating a tumor in a subject. The method comprises,
(a) administering to the subject an effective amount of a nanoparticle;
(b) applying ultrasound to the subject of the step (a) so as to produce an ultrasound image of the tumor, in which the location of the tumor is revealed by the ultrasound image; and
(c) administering to the subject a therapeutically effective amount of a radiation or an anti-tumor agent based on the location of the tumor revealed by the ultrasound image in the step (b).

Steps (a) and (b) are similar to those described in the first aspect of the present disclosure; hence, detailed description of these steps is omitted for the sake of brevity.

In the step (c), appropriate treatment is administered to the subject in accordance with the information of the tumor (including the boundary and the location of the tumor) revealed by the ultrasound image. The treatment may be any therapeutic approach exhibiting a cytotoxic and/or inhibitory effect on the tumor, for example, radiotherapy (i.e., the use of high doses of radiation to kill tumor cells), chemotherapy (i.e., suppressing tumor growth by using cytotoxic agent or anti-proliferative agent), anti-angiogenesis therapy (i.e., suppressing tumor growth via blocking the formation of new blood vessels, and thus, starving the tumor cells), immunotherapy (i.e., eliciting the immune response of the subject against tumor cells), targeting therapy (i.e., use of antibody for targeting a specific cellular marker), or hormone therapy (i.e., suppressing hormone-related tumors via blocking the synthesis, secretion, or function of hormone).

The agents commonly used in chemotherapy include, but are not limited to, doxorubicin, adriamycin, bleomycin, actinomycin, dactinomycin, mutamycin, daunorubicin, epirubicin, idarubicin, mitoxantrone, mitomycin, epipodophyllotoxins, etoposide, teniposide, antimicrotubule agent, vinblastine, vincristine, vindesine, vinorelbine, taxane, paclitaxel (taxol), docetaxel (taxotere), nitrogen mustard, chlorambucil, cyclophosphamide, estramustine, ifosfamide, mechlorethamine, melphalan, aziridines, thiotepa, alkyl sulfonate, busulfan, nitrosoureas, carmustine, lomustine, streptozocin, platinum complex, carboplatin, cisplatin, alkylator, altretamine, dacarbazine, procarbazine, temozolamide, methotrexate, fludarabine, mercaptopurine, thiogaunine, cladribine, pentostatin, capecitabine, cytarabine, floxuridine, fluorouracil, gemcitabine, hydroxyurea, camptothecin, irinotecan, busufane, epothilone, azathioprine, halofuginone, sirolimus, everolimus, mytomycin, and topotecan.

Exemplary anti-angiogenic agents include, but are not limited to, thrombospondin, angiostatin, pigment epithelium-derived factor (PEDF), endostatin, calreticulin, interferon-γ-inducible protein 10 (IP-10), platelet factor 4 (PF4), arresten, tumstatin, bevacizumab (Avastin®), cetuximab (Erbitux®), sunitinib (Sutent®), sorafenib (Nexavar®), tivozanib (AV-951), cediranib (AZD2171), dasatinib (Sprycel®), nilotinib (AMN-107), CP-547632, erlotinib (Tarceva®), panitumumab (Vectibix®), pazopanib (Votrient®), axitinib and gefitinib (Iressa®), and ranibizumab (Lucentis®).

Non-limiting examples of immunomodulatory agents include, thalidomide, lenolidomide, pomalidomide, anti-programmed cell death-1 (PD-1) antibody, anti-programmed cell death-ligand 1 (PD-L1) antibody, anti-cytotoxic T-lymphocyte-associated antigen 4 (CTLA-4) antibody, interleukin (IL)-2, IL-6, IL-12, interferon-alpha (IFN-α), IFN-γ, granulocyte-macrophage colony-stimulating factor (GM-C SF), granulocyte colony-stimulating factor (G-CSF), and cancer vaccine (e.g., human papillomavirus (HPV) vaccine, and hepatitis B vaccine).

The hormone therapy may be an androgen deprivation therapy (ADT; also known as androgen suppression therapy), an estrogen deprivation therapy (EDT; also known as endocrine therapy), or high-dose estrogen (HDE). Examples of anti-hormone agents used in ADT include, but are not limited to, leuprorelin, goserelin, triptorelin, histrelin, buserelin, degarelix, cyproterone acetate, flutamide, nilutamide, bicalutamide, enzalutamide, abiraterone acetate, seviteronel, enzalutamide, apalutamide, and darolutamide. Non-limiting examples of anti-hormone agents used in EDT include, tamoxifen, toremifene, raloxifene, ospemifene, bazedoxifene, fulvestrant, brilanestrant, elacestrant, anastrozole exemestane, and letrozole. The estrogens used in HED include, but are not limited to, conjugated estrogen, estradiol, estradiol ester (such as estradiol benzoate, estradiol undecylate, estradiol valerate, and polyestradiol phosphate), estramustine phosphate, ethinylestradiol, mestranol, ethinylestradiol sulfonate, diethylstilbestrol, fosfestrol, and bifluranol.

Targeting therapy include trastuzumab or pertuzumab (an antibody specific to tumor antigen HER-2/neu); bevacizumab (an antibody specific to vascular endothelial growth factor (VEGF)); ramucirumab (an antibody specific to VEGF receptor); nivolumab or cemiplimab (an antibody specific to programmed cell death protein 1 (PD-1)); atezolizumab, avelumab or durvalumab (an antibody specific to the ligand of programmed cell death protein 1 (PD-L1), Ipilimumab (an antibody specific to cytotoxic T-lymphocyte-associated protein 4 (CTLA-4)), rituximab (an antibody specific to CD20 on B cells), and etc.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

### Example 1 Effect of Nanoparticles in Enhancing Contrast of an Ultrasound Image

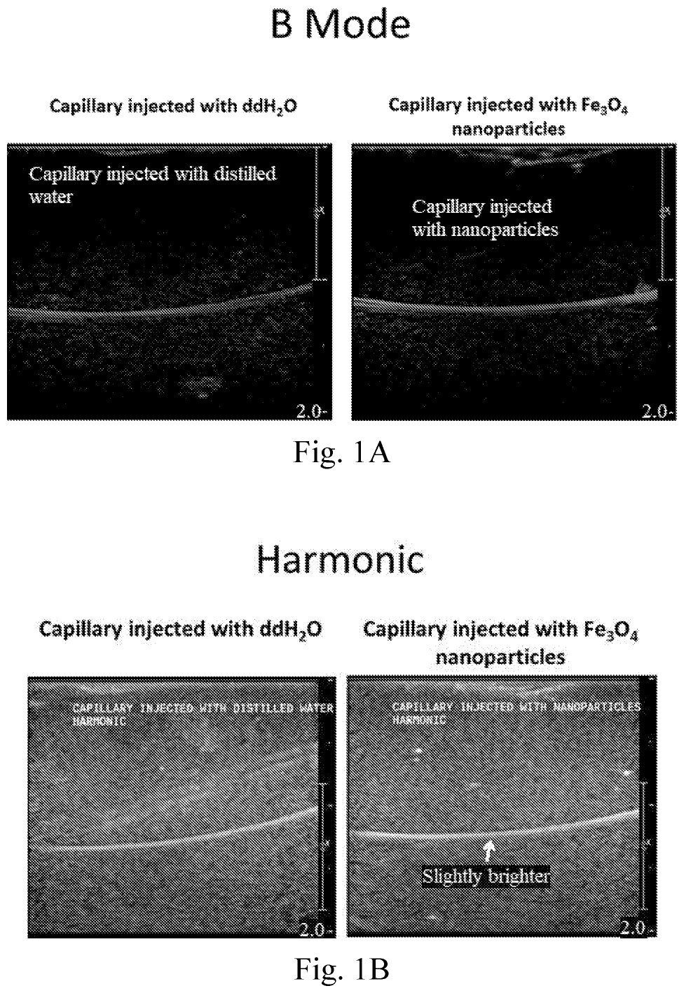

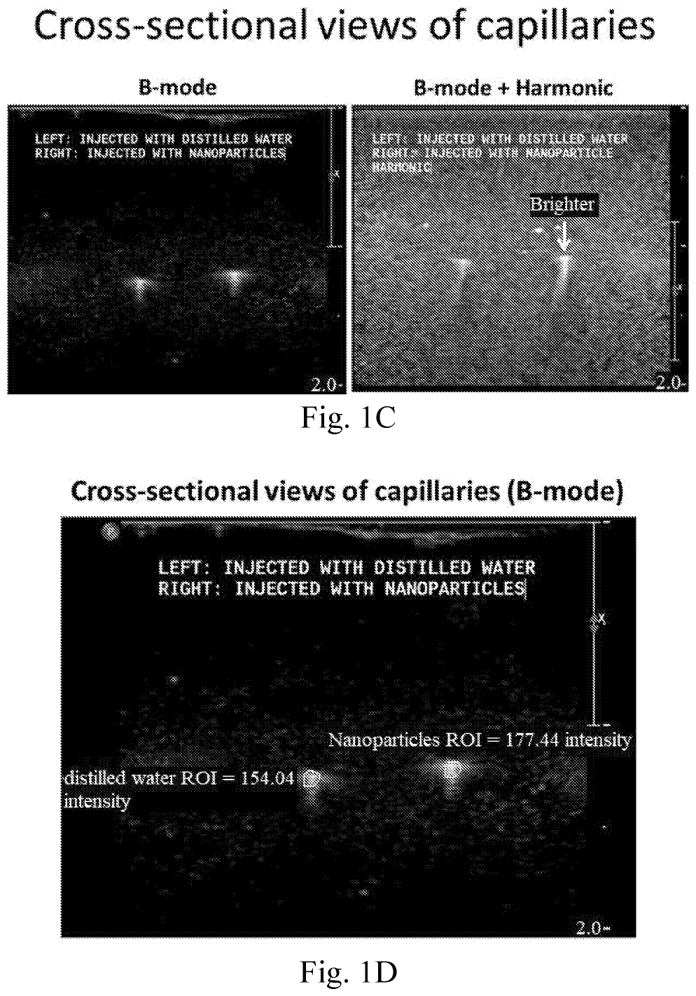

In this example, the effect of nanoparticles of the present disclosure in improving ultrasound images of a tumor was evaluated by phantom gels, in which agarose or agar was used to simulate solid tumors. The results were respectively depicted in FIGS. 1-4.

1.1 Phantom Gel Prepared from Agarose

The agarose phantom gel was prepared by adding sephadex (4 g) into a container containing agarose (7.5 g), distilled water (200 ml) and glycerol (30 ml) under gently stirring, so as to prevent the mixture from clumping together. The mixture was then heated and boiled for about 2 to 5 minutes. The boiled mixture was then poured into a plastic container, and left in ventilation hood at room temperature for about 20 minutes to prevent formation of air bubbles in the mixture. Two capillaries (each with a diameter of 0.1 mm) was inserted into the phantom gel when it started to solidify but not yet totally turned into its solid form. Distilled water (serving as the control group) and the solution containing Fe₃O₄ nanoparticles (size: about 30 nm; concentration: 5 mg/ml) were respectively injected into two capillaries by syringe to fill the lumens of capillaries. The phantom gel was then kept at 4° C. for 1 to 2 hours until it was completely solidified.

The phantom gel was examined by an ultrasound imaging system (Philips iU22), using transducer L15-7ᵢₒ (composite frequency ranging from 7 to 15 MHz), with musculoskeletal superficial option. The image of phantom gel was captured by B mode or harmonic mode. The captured images were then analyzed by quantitative analysis software, using region of interest (ROI) analysis option to measure the echo intensity of the phantom gel with or without nanoparticles.

The data in FIGS. 1A-1D indicated that a capillary having an inner diameter (i.d.) of 0.1 mm was visible by ultrasound imaging, in which the size of the capillary on the ultrasound image was about 0.4 mm that was close to the limit of ultrasound resolution at this frequency (i.e., 7 to 15 MHz). The signal intensity of the capillary filled with the iron oxide magnetic nanoparticle was obviously higher than that of the capillary filled with distilled water (FIGS. 1A-1D). Taken together, the results in this example demonstrated that the magnetic nanoparticles were capable of enhancing the signal intensity of ultrasound imaging.

1.2 Phantom Gel Prepared from Agar

The agar phantom gel was prepared by mixing agar (3.7 g), isopropanol (8 ml), and distilled water (92 ml), and heated the mixture for about 2 to 5 minutes until it boiled. The boiled agar gel was then poured into a container at room temperature and let cool. When the liquid agar gel started to solidify but still in the liquid form, 6.3 microliters of double distilled water (serving as the control group) and a silver nanoparticle solution (particle size: 80 nm; concentration: 8.8×10⁸ particles/ml) were respectively injected into two different locations of the agar phantom by syringe. After injection, the phantom was left at room temperature until it was completely solidified, and then stored at 4° C. for 1 hour.

The phantom gel was examined by an ultrasound imaging system (Philips iU22), using transducer L12-5 (composite frequency ranging from 5 to 12 MHz), with musculoskeletal superficial option. The image of phantom gel was captured by B mode.

As the result in FIG. 2 indicated, compared with the control group, where no signal was detected by ultrasound imaging, the ultrasound energy reflected by silver nanoparticles, and thus emitted an echo signal on the ultrasound image confirmed the presence of the silver nanoparticles, in which the size of each silver nanoparticle on the ultrasound image was about 0.5 mm, which was similar to the size of the tumor/cancer at stage 0 or stage 1, e.g., an early-stage considered curable in clinical practice.

1.3 Artificial Tumors Embedded in Tissue-Mimicking Phantom Gel

The tissue-mimicking phantom gel having artificial tumors embedded therein was prepared by the following procedures.

(i) Preparation of Artificial Tumors

A mixture of ddH₂O (40 ml), agarose (1 g), and all-purpose flour (0.5 g) was boiled, and then poured into a 10-cm dish and let cool. Air bubbles were removed by a pin or moved to the wall of the dish. The mixture was left at room temperature until it was completely solidified. The solidified phantom gel was then stored at 4° C. for 1 hour before use. After one hour, two artificial tumors were made from the solidified phantom gel using a mold. Each artificial tumor was cylindrical and had the size of π×(1 mm)²×2 mm. The artificial tumors were independently immersed in 10 μl of ddH₂O, and the solution containing iron oxide (II, III) nanoparticles (i.e., Fe₃O₄ nanoparticles; 30 nm avg. part. size under transmission electron microscopy (TEM), 5 mg/mL in H₂O) at room temperature for 3 hours.

(ii) Preparation of Tissue-Mimicking Phantom Gel

Tissue-mimicking phantom was prepared by boiling a mixture of ddH₂O (40 mL), agarose (1 g), and all-purpose flour (0.5 g). The boiled mixture was then poured into a 10-cm dish and let cool. Air bubbles were removed by a pin or moved to the wall of Petri dish. Artificial tumors prepared in the step (i) were inserted into the liquid mixture before it solidified. The phantom gel was left at room temperature until it was completely solidified. The solidified phantom gel was then stored at 4° C. for 1 hour.

The thus-prepared solidified phantom gel was examined by a diagnostic ultrasound system, using transducer L154BH (frequency: 12 MHz), with musculoskeletal option, and the obtained image was captured by B mode.

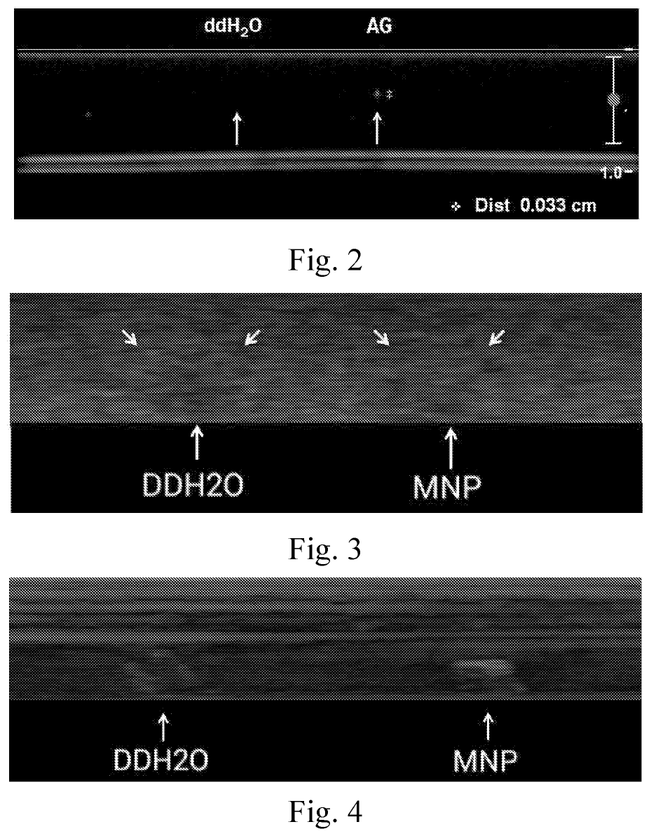

As depicted in FIG. 3, the artificial tumor soaked in ddH₂O appeared to be hypoechoic as compared to the background, while the border of the artificial tumor soaked in the solution containing Fe₃O₄ nanoparticles could be clearly observed by the ultrasound examination.

1.4 Artificial Tumors Made of 2% Agar and Embedded in 2% Agar Phantom

The 2% agar gel having artificial tumors embedded therein was prepared by the following two steps.

(i) Preparation of Artificial Tumors

A mixture containing ddH₂O (40 ml) and agar powder (0.8 g) was boiled, and then poured into a 10-cm dish and let cool. The mixture was left at room temperature until it was completely solidified. The solidified phantom gel was then stored at 4° C. for 1 hour before use. Later, two artificial tumors were made from the solidified phantom gel using a mould. Each artificial tumor was cylindrical and had the size of π×(1 mm)²×2 mm. The artificial tumors were respectively immersed in 10 μl of ddH₂O and the solution containing Fe₃O₄ nanoparticles (30 nm avg. part. size under TEM, 5 mg/mL in H₂O) at room temperature for 3 hours.

(ii) Preparation of 2% Agar Phantom Gel

2% agar phantom was prepared by boiling a mixture of ddH₂O (40 ml) and agar (0.8 g). The boiled mixture was then poured into a 10-cm dish and let cool. Artificial tumors were inserted into the liquid mixture before it was solidified. The phantom gel was left at room temperature until it was completely solidified. Next, the solidified phantom was stored at 4° C. for 1 hour before use.

The solidified phantom gel was examined by a diagnostic ultrasound system, using transducer L154BH (frequency: 12 MHz), with musculoskeletal option, and the obtained image was captured by B mode.

The data of FIG. 4 indicated that the artificial tumor soaked in ddH₂O was hypoechoic under the ultrasound examination, while the artificial tumor soaked in the solution containing Fe₃O₄ nanoparticles exhibited a clear edge in the ultrasound image, and appeared to be hyperechoic under the ultrasound examination.

### Example 2 Effect of Nanoparticles in Enhancing Contrast of an Ultrasound Image of a Biological Sample

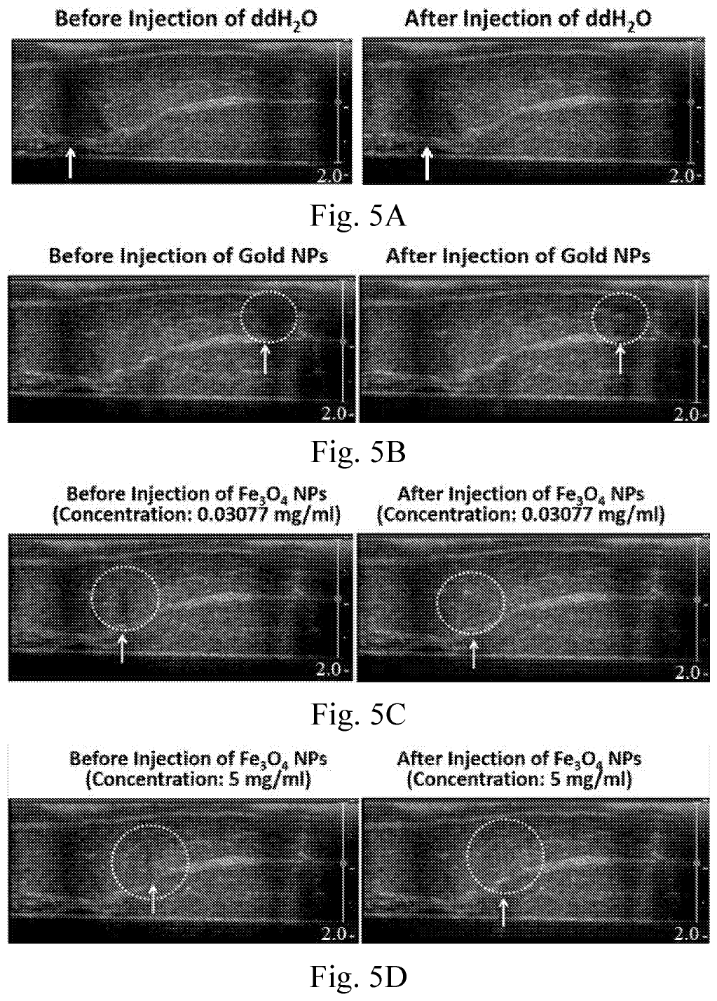

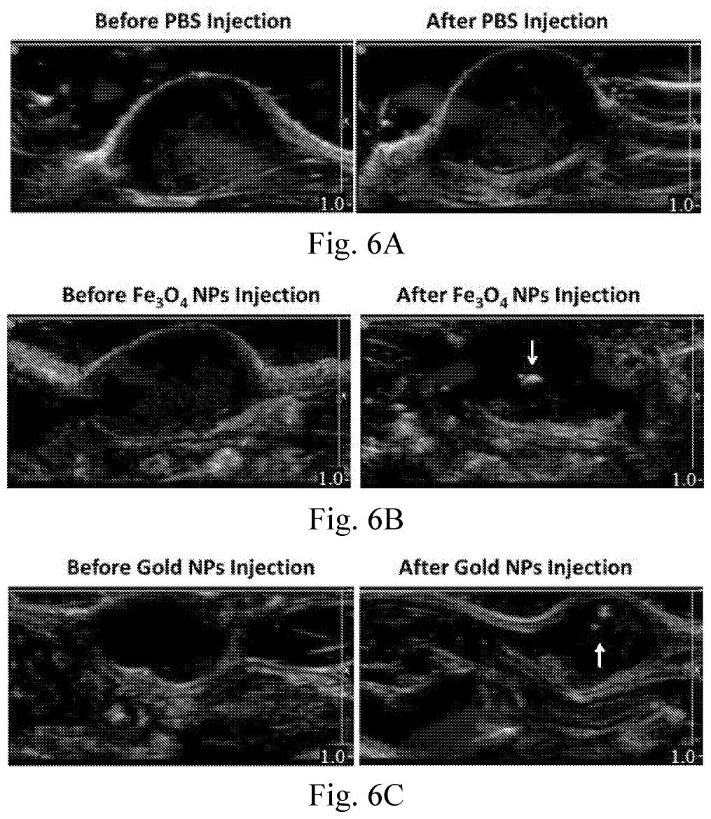

The effect of nanoparticles in enhancing ultrasound images of biological samples were further investigated in this example. The results are depicted in FIGS. 5A-5D.

A piece of pork was first washed with PBS, then was placed on top of Fe₃O₄ nanoparticles. 20 μl of ddH₂O, gold nanoparticles (corpuscular gold nanospheres, diameter: 20 nm, number: 7×10¹¹ particles/ml or concentration: 0.03077 mg/ml), or Fe₃O₄ nanoparticles (average diameter: 20 nm, concentration: 0.03077 mg/ml or 5 mg/ml) were injected into different portions of the pork. Images before and after the injection were taken by B mode of Philips iU22 ultrasound imaging system, using transducer L12-5 (composite frequency ranged from 5 to 12 MHz), with musculoskeletal superficial option.

As depicted in FIGS. 5A-5D, no signal was detected at the ddH₂O-injected site (FIG. 5A), while the signal intensity of the ultrasound image increased at the site injected with gold nanoparticles (FIG. 5B) or Fe₃O₄ nanoparticles (FIGS. 5C and 5D), accordingly, a bright spot was observed at the nanoparticle-injected site, which was marked by the circle in FIGS. 5B-5D. These results demonstrated that the gold nanoparticle and the Fe₃O₄ nanoparticle were both useful in enhancing the signal intensity of ultrasound image.

### Example 3 In Vivo Analysis

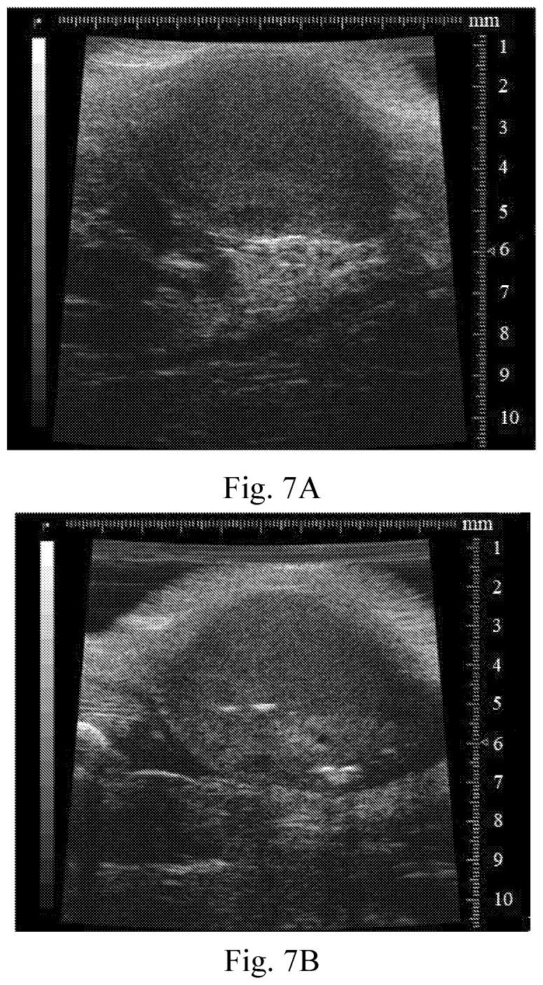

Whether the nanoparticle would enhance the accuracy and sensitivity of ultrasound-based tumor diagnosis in animals was examined in this example. The results were depicted in FIGS. 6 and 7.

3.1 Lung Cancer Model

1×10⁶ of CL1-5-GL cells (a human lung cancer cell line) were injected subcutaneously into mice. Three weeks later, 80 μl of PBS (serving as the control group), Fe₃O₄ nanoparticles (30 nm avg. part. size under TEM, carboxylic acid functionalized, dispersed in PBS, 10 mg/mL), or gold nanoparticles (30 nm; dispersed in PBS, 10 mg/ml) were intratumorally injected to the tumor-bearing mice. Images of the tumor were taken before and after injection by Philips IU-22 ultrasound imager, using transducer L12-5 (composite frequency ranged from 5 to 12 MHz), with musculoskeletal superficial option. The images were captured by B mode.

The data of FIG. 6A indicated that no difference was detected in the tumor before (FIG. 6A, the left photograph) and after (FIG. 6A, the right photograph) PBS injection. Compared with the tumor without nanoparticle injection (FIGS. 6B and 6C, the left photographs), localized brighter area was observed in the tumor injected with Fe₃O₄ nanoparticles (FIG. 6B, the right photograph) or gold nanoparticles (FIG. 6C, the right photograph). The results of FIGS. 6A-6C demonstrated that the injection of the Fe₃O₄ nanoparticle (FIG. 6B, the right photograph) or the gold nanoparticle (FIG. 6C, the right photograph) may increase the sensitivity of the ultrasound-based tumor imaging in animals.

3.2 Breast Cancer Model

5×10⁶ of MDA-MB-231 cells was subcutaneously injected into mouse. When the tumor grew up to 5×5×5 mm³, 100 μl of magnetic nanoparticle (MNP, Fe₃O₄ nanoparticles) (5 mg/ml) was intratumorally injected into the tumor-bearing mice. Images of the tumor were taken before and after injection by VEVO 770 ultrasound imager, using transducer L12-5 (composite frequency ranged from 5 to 12 MHz), with musculoskeletal superficial option. The images were captured by B mode.

As the results depicted in FIGS. 7A and 7B, after the injection of the Fe₃O₄ nanoparticle, the edge of the tumor may be observed more clearly as compared with the control group (i.e., the tumor without nanoparticle injection).

In conclusion, the present disclosure provides a novel use of nanoparticles (including the gold, silver, and iron oxide nanoparticles) in enhancing the accuracy and/or sensitivity of ultrasound-based tumor imaging, and accordingly, enabling a skilled artisan to make a diagnosis of tumors, especially the early-stage, in a more accurate and precise manner.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of enhancing the accuracy and/or sensitivity of ultrasound imaging in detecting a tumor in a subject, comprising:
(a) administering to the subject an effective amount of a nanoparticle; and
(b) applying ultrasound to the subject of the step (a) to produce an ultrasound image of the tumor and a neighboring normal tissue, wherein the nanoparticle increases the contrast between the tumor and the normal tissue on the ultrasound image thereby enhancing the accuracy and/or sensitivity of ultrasound imaging in detecting the tumor.

2. The method of claim 1, wherein the nanoparticle is a metal nanoparticle, a metal oxide nanoparticle, a silicon nanoparticle, or a silicon oxide nanoparticle.

3. The method of claim 2, wherein the metal nanoparticle is a silver (Ag) nanoparticle, a gold (Au) nanoparticle, a platinum (Pt) nanoparticle, a palladium (Pd) nanoparticle, a copper (Cu) nanoparticle, a cobalt (Co) nanoparticle, a chromium (Cr) nanoparticle, a nickel (Ni) nanoparticle, an iron (Fe) nanoparticle, a titanium (Ti) nanoparticle, an aluminum (Al) nanoparticle, a lead (Pb) nanoparticle, a rhodium (Rh) nanoparticle, a tantalum (Ta) nanoparticle, a ruthenium (Ru) nanoparticle, a tungsten (W) nanoparticle, a gadolinium (Gd) nanoparticle, or an alloy thereof.

4. The method of claim 2, wherein the metal oxide nanoparticle is a zinc oxide (ZnO) nanoparticle, a magnesium oxide (MgO) nanoparticle, a manganese oxide (Mn₂O₃) nanoparticle, a magnetite (Fe₃O₄) nanoparticle, a maghemite (γ-Fe₂O₃) nanoparticle, a cobalt ferrite (CoFe₂O₄) nanoparticle, a manganese ferrite (MnFe₂O₄) nanoparticle, or a strontium iron oxide (SrFe₁₂O₁₉) nanoparticle.

5. The method of claim 1, wherein the diameter of the nanoparticle ranges from 1 nanometer to 999 nanometers.

6. The method of claim 1, wherein the nanoparticle further comprising an antibody or a fragment thereof, an aptamer, a targeting peptide conjugated thereto.

7. The method of claim 1, wherein the nanoparticle is administered in a solution, an emulsion, or a gel.

8. The method of claim 7, wherein the nanoparticle is administered in the solution, and the nanoparticle is present in the solution at a concentration of 1 mg/ml to 100 mg/ml.

9. The method of claim 8, wherein the solution is administered to the subject in a volume of 0.01 ml/Kg to 50 ml/Kg body weight of the subject.

10. A method of treating a tumor in a subject, comprising,
(a) administering to the subject an effective amount of a nanoparticle;
(b) applying ultrasound to the subject of the step (a) to produce an ultrasound image of the tumor, in which the location of the tumor is revealed by the ultrasound image; and
(c) administering to the subject a therapeutically effective amount of a radiation or an anti-tumor agent based on the location of the tumor revealed by the ultrasound image in the step (b).

11. The method of claim 10, wherein the nanoparticle is a metal nanoparticle, a metal oxide nanoparticle, a silicon nanoparticle, or a silicon oxide nanoparticle.

12. The method of claim 11, wherein the metal nanoparticle is a silver (Ag) nanoparticle, a gold (Au) nanoparticle, a platinum (Pt) nanoparticle, a palladium (Pd) nanoparticle, a copper (Cu) nanoparticle, a cobalt (Co) nanoparticle, a chromium (Cr) nanoparticle, a nickel (Ni) nanoparticle, an iron (Fe) nanoparticle, a titanium (Ti) nanoparticle, an aluminum (Al) nanoparticle, a lead (Pb) nanoparticle, a rhodium (Rh) nanoparticle, a tantalum (Ta) nanoparticle, a ruthenium (Ru) nanoparticle, a tungsten (W) nanoparticle, a gadolinium (Gd) nanoparticle, or an alloy thereof.

13. The method of claim 11, wherein the metal oxide nanoparticle is a zinc oxide (ZnO) nanoparticle, a magnesium oxide (MgO) nanoparticle, a manganese oxide (Mn₂O₃) nanoparticle, a magnetite (Fe₃O₄) nanoparticle, a maghemite (γ-Fe₂O₃) nanoparticle, a cobalt ferrite (CoFe₂O₄) nanoparticle, a manganese ferrite (MnFe₂O₄) nanoparticle, or a strontium iron oxide (SrFe₁₂O₁₉) nanoparticle.

14. The method of claim 10, wherein the diameter of the nanoparticle ranges from 1 nanometer to 999 nanometers.

15. The method of claim 10, wherein the nanoparticle further comprising an antibody or a fragment thereof, an aptamer, a targeting peptide conjugated thereto.

16. The method of claim 10, wherein the nanoparticle is administered in a solution, an emulsion, or a gel.

17. The method of claim 16, wherein the nanoparticle is administered in the solution, and the nanoparticle is present in the solution at a concentration of 1 mg/ml to 100 mg/ml.

18. The method of claim 17, wherein the solution is administered to the subject in a volume of 0.01 ml/Kg to 50 ml/Kg body weight of the subject.

19. The method of claim 10, wherein the tumor is selected from the group consisting of melanoma, tongue carcinoma, colorectal carcinoma, esophageal carcinoma, gastric carcinoma, lung cancer, bladder cancer, breast cancer, pancreatic cancer, renal cancer, hepatocellular carcinoma, ovarian cancer, prostate cancer, and head and neck squamous cell carcinoma.

20. The method of claim 10, wherein the anti-tumor agent is a chemotherapeutic agent, an anti-proliferative agent, an anti-angiogenic agent, an immunomodulatory agent, or an anti-hormone agent.
